(19) United States
(12) Patent Application Publication
(10) Pub. No.: US 2009/0018482 A1
(43) Pub. Date: Jan. 15, 2009

(54) Sock

(76) Inventor: Bodo W. Lambertz, Pfaffikon (CH)

Correspondence Address: Olson & Cepuritis, LTD., 20 NORTH WACKER DRIVE, 36TH FLOOR, CHICAGO, IL, 60606, US

(21) Appl. No.: 12/087,043

(22) PCT Filed: Jan. 10, 2007
(86) PCT No.: PCT/DE2007/000019

§ 371 Date: Jun. 24, 2008

(30) Foreign Application Priority Data
Jan. 27, 2006 (DE) 20 2006 001 379.2

(51) Int. Cl. A61F 13/06; A41B 11/00

(52) U.S. Cl. 602/65; 2/239

(57) ABSTRACT

The invention relates to a sock, especially for use in sports activities. Said sock consists of a shaft and a foot part having a toe and a heel area and a contact area between the toe and heel area, and is provided with a bandage. Said bandage (3) has a conical shape.

The invention relates to a sock, in particular, for use in athletic activities, having a shaft and a foot part, which has a toe area and a heel area and a tread area situated between the toe area and the heel area, and is provided with a bandage.

In athletic activities, in particular, the human foot is usually surrounded by socks. To achieve a good fit of the sock to the foot, it is known that ring bandages may be provided on socks or stockings.

These are each provided with a tubular cross section. The so-called bandages serve to support the joint in athletic activities. They usually fulfill the requirements made of them. However, the bandages do not take into account the essentially narrow cross section of the foot in the area above the ankle and how it widens in the direction of the foot. Consequently there is the possibility that the part of the bandage facing the foot might fit too tightly in the area of the foot that widens because of having the same diameter as the part facing away from the foot. This may lead to pressure points. Since the socks are worn directly against the skin, this may also lead to skin abrasions. In any case the efficiency of the athlete is thereby reduced.

This is where the present invention wants to create an expedient. The object of the invention is to create a sock which takes into account, in particular, the anatomy of the foot and is optimally adapted to the given contours in the transition between the foot and the shin. According to the invention, this object is achieved by the fact that the bandage has a conical design.

The present invention creates a sock for athletic activities, in particular, having a funnel-shaped design and taking into account, in particular, the anatomical conditions in the transition between the shin and the foot. This avoids pressure points in the area of the end of the bandage facing the foot. Furthermore the design of the bandage causes it to be in tighter contact with the ankle during athletic activity so this additionally increases the protective function. At the same time the bandage prevents slippage of the shaft in the direction of foot.

In a further embodiment of the invention, the bandage forms the closure of the shaft on its side facing away from the tread area. This creates the possibility of avoiding the band that is usually provided on socks. This is especially advantageous with very short socks such as running socks, in particular, or tennis socks. This also takes into account the prevailing trends in the different types of sports, while at the same time achieving a high supporting function.

In another refinement of the invention, a web is provided. The web preferably extends essentially from the area of the ankle in the direction of the tread area, most preferably being connected to the bandage at its free ends. Providing the web leads to a further fixation of the bandage and thereby to an even better fit of the sock on the foot. Due to the essentially U-shaped design of the web under the tread area, this also produces an increased lateral supporting function.

Other refinements and embodiments of the invention are characterized in the other subordinate claims. Exemplary embodiments of the invention are depicted in the drawings and are described in detail below, where:

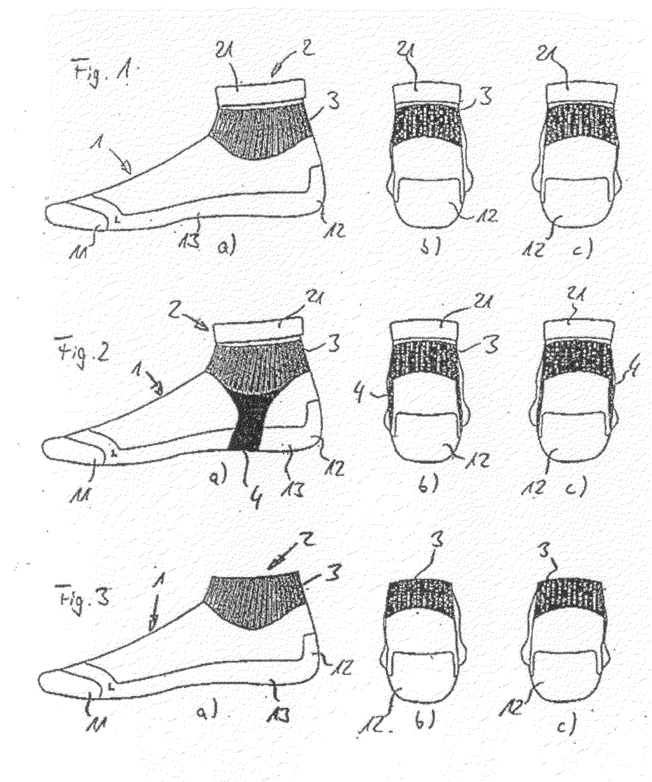

FIG. 1a) to c) show a sock with a bandage in the view of the outside of the foot and the view of the pair of socks from the rear;

FIG. 2a) to c) show a sock with a bandage in the view of the outside of the foot and the view of the pair of socks from the rear in another embodiment and

FIG. 3a) to c) show a sock with a bandage in the view of the outside of the foot and a view of the pair of socks from the rear in another embodiment.

The sock selected as the exemplary embodiment comprises a foot part and a shaft 2. The foot part 1 has a toe area 11, a heel area 12 and a tread area 13 situated between the toe area and the heel area. The areas 11, 12 and 13 may be made of a reinforced material, as depicted in the exemplary embodiment. Use of material combinations such as sheared wool with elastomer fiber materials is also possible. The shaft 2 is provided with a band 21 on its end facing the foot part 1 in the exemplary embodiments according to FIGS. 1 and 2.

In the area of the ankle, the sock is provided with a bandage 3. The bandage 3 is made of an elastic and climate regulating woven material. Elastan, Lycra or other materials of different elasticity are preferred here.

The bandage 3 runs continuously around the sock and is woven with the fabric forming the sock. It has a conical design and therefore differs in diameter over its width. On the side facing away from the tread area 13, the bandage 3 has a smaller diameter than on the side facing the tread area 13, i.e., facing away from the band 21. This results in a funnel shape design of the bandage 3. Due to this design, the bandage 3 sits tightly against the ankle during athletic activity, so the supporting effect is additionally reinforced. At the same time, this prevents the sock from slipping in the direction of the foot part 1 during athletic activity, which thereby increases the wearing comfort.

In the exemplary embodiment according to the FIG. 2, a web 4 is provided. The web 4 extends essentially from the area of the ankle in the direction of the tread area 13 and runs through it across the longitudinal center line of the tread area 13. The web 4 therefore surrounds the foot from the ankle of the inside to the ankle on the outside by surrounding the foot in this area—in a U shape as seen from the rear. With its free ends the web 4 is connected to the bandage 3. The web 4 produces an additional fixation of the bandage 3 and leads to an even more accurate fit of the sock. Although the web 4 exerts a force on the bandage in the direction of the tread area 13, the bandage 3 remains in its predetermined position due to its conical design.

In the exemplary embodiment according to FIG. 3, the sock does not have a band. The bandage 3 here assumes the function of the band. The bandage 3 therefore forms the closure of the shaft 2 on its side facing away from the tread area 13. This embodiment is recommended, in particular, for short running socks, tennis socks and the like.

When speaking of socks in the description and the claims, the invention is not limited to these alone but instead this term is also meant to subsume stockings, panty hose and the like, to which the invention also refers.

1. A sock, in particular, for use in athletic activities, having a shaft and a foot part which has a toe area and a heel area and a tread area situated between the toe area and the heel area and is provided with a bandage, characterized in that the bandage is provided in the area of the ankle and has a conical design.

2. The sock according to claim 1, characterized in that the bandage (3) has a smaller diameter on its side facing away from the tread area (13) than on the side facing the tread area (13).

3. The sock according to claim 1, characterized in that the bandage (3) forms the closure of the shaft (2) on the side facing away from the tread area (13).

4. The sock according to claim 1, characterized in that a web (4) is provided.

5. The sock according to claim 4, characterized in that the web (4) extends essentially from the area of the ankle in the direction of the tread area (13).

6. The sock according to claim 4, characterized in that the web (4) is connected at its free ends to the bandage (3).

7. The sock according to claim 2, characterized in that the bandage (3) forms the closure of the shaft (2) on the side facing away from the tread area (13).

8. The sock according to claim 2, characterized in that a web (4) is provided.

9. The sock according to claim 3, characterized in that a web (4) is provided.

10. The sock according to claim 5, characterized in that the web (4) is connected at its free ends to the bandage (3).
